(19) United States
(12) Patent Application Publication
(10) Pub. No.: US 2026/0216107 A1
(43) Pub. Date: Jul. 30, 2026

(54) INTERLEUKIN-6 (IL-6)/SIGNAL TRANSDUCER AND ACTIVATOR OF TRANSCRIPTION 3 (STAT3) PATHWAY INHIBITORS FOR USE IN THE TREATMENT OF GRANULOMATOUS DISEASES

(71) Applicant: UCL BUSINESS LTD, London Greater London (GB)

(72) Inventors: Alan David SALAMA, London Greater London (GB); Scott Russell HENDERSON, London Greater London (GB)

(73) Assignee: UCL BUSINESS LTD, London Greater London (GB)

(21) Appl. No.: 19/151,669

(22) PCT Filed: Jan. 30, 2024
(86) PCT No.: PCT/GB2024/050254

§ 371 Date: Jul. 29, 2025

(30) Foreign Application Priority Data
Jan. 30, 2023 (GB) 2301306.3

(51) Int. Cl. A61K 31/167; A61P 37/02; C07K 16/24

(52) U.S. Cl. CPC A61K 31/167; A61P 37/02; C07K 16/248

(57) ABSTRACT

The invention relates to the treatment of granulomatous diseases, and in particular the use of niclosamide, an IL-6 inhibitor, a STAT3 inhibitor, and/or an IL-6/STAT3 pathway inhibitor, in the treatment of a granulomatous disease.

### TECHNICAL FIELD

The invention relates to the treatment of granulomatous diseases, and in particular the use of niclosamide, an IL-6 inhibitor, a STAT3 inhibitor and/or an IL-6/STAT3 pathway inhibitor in the treatment of a granulomatous disease.

### INTRODUCTION

Granulomatous diseases refers to a group of diseases characterised by the presence of granulomas. Granulomas are formed when macrophages aggregate and form a mass. Macrophages that form granulomas are known as histiocytes and can fuse together in a tight ball and may form giant cells, known as a Langerhans giant cell or a multinucleate giant cell. Granulomas may also comprise cells including lymphocytes, neutrophils, eosinophils and/or fibroblasts. Granulomas may also comprise collagen.

Granulomas often arise in response to chronic inflammation, they may form in response to an antigen that cannot be immediately neutralised by the immune system or as a result of an autoimmune disease.

Examples of autoimmune granulomatous diseases include Crohn's disease, antineutrophil cytoplasmic antibodies (ANCA) associated vasculitis and sarcoidosis. Granulomatous diseases are typically treated with long term, high toxicity immune suppressants, and/or steroids. These treatments are often inadequate as the granulomas are difficult to resolve and may take a prolonged period of time to respond, meaning that patients are exposed to augmented doses of therapy which lead to excessive adverse effects. This has a negative impact on patients' quality of life, as well as having an associated significant healthcare burden.

For example, granulomatosis polyangiitis (GPA), a form of ANCA associated vasculitis, is a chronic frequently relapsing autoimmune disease with a prevalence of 200/million population. <950 people are diagnosed with GPA each year in England, and there are about 11,400 people currently living with these conditions. The incidence of ANCA-associated vasculitis increases with age and the peak age of onset is between 60 and 70 years.

GPA is characterised by blood vessel inflammation leading to multi-organ involvement and frequently results in kidney failure necessitating dialysis or transplantation, making it a significant healthcare issue. Due to its relapsing nature, patients require prolonged courses of expensive immunosuppressive treatment which result in many adverse events, particularly infections, leading to recurrent hospitalisations. Within the first year from diagnosis, patients with ANCA associated vasculitis are admitted 8 times more often than age- and gender-matched populations (10 times more often to ICU's), similar to COPD patients. Although this rate declines with time, it remains more than double that of matched populations 10 years after diagnosis, with average lengths of stay are 2.5 times greater than non-ANCA associated vasculitis patients. Patient-related healthcare costs double following a diagnosis of ANCA associated vasculitis, while poor disease control in the first year leads to an almost trebling of costs. In Scotland, costs for ANCA associated vasculitis patients are 4 times greater than for non-ANCA associated vasculitis patients, even without taking into account those requiring dialysis and the cost of drugs such as rituximab, emphasizing the high healthcare burden of the disease. Hence, preventing infections and severe adverse events related to therapy would have a significant impact not only on patients' quality of life but also on healthcare costs. There is therefore a clear and significant unmet clinical need for improved treatments for granulomatous diseases, such as GPA.

The present invention aims to address or ameliorate one or more of the issues associated with current therapies for granulomatous diseases.

### SUMMARY OF THE INVENTION

According to a first aspect, the invention provides an interleukin-6 (IL-6)/signal transducer and activator of transcription 3 (STAT3) pathway inhibitor for use in treating a granulomatous disease in a subject.

In another aspect, the invention provides a method of treating a granulomatous disease in a subject comprising administering an IL-6/STAT3 pathway inhibitor to the subject.

The IL-6/STAT3 pathway inhibitor may inhibit any point on the IL-6/STAT3 pathway.

The IL-6/STAT3 pathway inhibitor in any aspect of the invention may be one or more of: an IL-6 inhibitor; a STAT3 inhibitor; niclosamide or a salt thereof; and an anti-IL-6 antibody or fragment thereof.

In a further aspect, the invention provides niclosamide or a salt thereof for use in treating a granulomatous disease in a subject.

Another aspect of the invention provides an anti-IL-6 antibody or fragment thereof for use in treating a granulomatous disease in a subject.

In a yet further aspect the invention provides a method of treating a granulomatous disease in a subject comprising administering a) niclosamide or a salt thereof or b) an anti-IL-6 antibody or fragment thereof to the subject.

In all aspects the granulomatous disease may be Antineutrophil Cytoplasmic Antibodies (ANCA) associated vasculitis, Crohn's disease, or sarcoidosis. The ANCA associated vasculitis may be granulomatosis with polyangiitis or eosinophilic granulomatosis with polyangiitis.

Another aspect of the invention provides an IL-6 inhibitor for use in treating a granulomatous disease in a subject.

A further aspect of the invention provides a STAT3 inhibitor for use in treating a granulomatous disease in a subject.

Another aspect of the invention provides a method of treating a granulomatous disease in a subject by administering an IL-6 inhibitor and/or a STAT3 inhibitor to a subject.

A further aspect of the invention provides an IL-6 inhibitor, a STAT3 inhibitor, an inhibitor of the IL-6/STAT3 pathway, niclosamide or a salt thereof, and/or an anti-IL-6 antibody or fragment thereof, for use in the manufacture of a medicament for treating a granulomatous disease in a subject.

The subject may be a mammal, and preferably, the subject is human.

The granulomatous disease may be ANCA associated vasculitis, Crohn's disease, or sarcoidosis.

The ANCA associated vasculitis may be granulomatosis with polyangiitis (GPA) or eosinophilic granulomatosis with polyangiitis.

The IL-6 inhibitor, the STAT3 inhibitor, and/or the IL-6/STAT3 pathway inhibitor, may be niclosamide or a salt thereof.

The IL-6 inhibitor, the STAT3 inhibitor, and/or the IL-6/STAT3 pathway inhibitor, may be a monoclonal antibody.

### Granulomatous Disease

As discussed above there are a number of diseases that are associated with granuloma, and thus are referred to as granulomatous diseases, including the following.

ANCA associated vasculitis which encompasses granulomatosis with polyangiitis (GPA), previously known as Wegener's granulomatosis, and eosinophilic granulomatosis with polyangiitis (EGPA). Each of these diseases may be associated with anti-neutrophil cytoplasmic antibodies (ANCAs).

GPA is a condition in which blood vessels become inflamed, and primarily affects the ears, nose, sinuses, kidneys and lungs. GPA can affect people of any age including children, but is more common in older people. There is no cure for the disease, it is currently managed using medication including immunosuppressants and steroids. Drugs typically used in the treatment of GPA are cyclophosphamide, rituximab, mycophenolate mofetil, methotrexate and steroid tablets. These are all associated with significant side effects. Initial treatment will last for up to around 5 years, once symptoms are under control, treatment may stop, but there is a high chance of disease relapse.

EGPA is characterised by inflammation of the small blood vessels which can cause organ damage throughout the body. The disease develops through three stages, stage one presents as adult onset asthma, the second stage is characterised by excess eosinophils, while the third stage progresses to vasculitis. The treatment of EGPA is similar to GPA and involves several years of steroids and immunosuppressants.

Crohn's disease is a form of inflammatory bowel disease with no known cure. Intestinal granulomas are used for microscopic diagnosis of Crohn's disease. Disease management is done via diet and lifestyle changes, and medication for symptoms. Crohn's disease may be associated with granulomatous ulcers inside the mouth, granulomatous hepatitis, granulomatous lung disease, and intestinal granulomas.

Sarcoidosis is a rare disease associated with the formation of granulomas. Sarcoidosis most commonly affects the lungs, lymph nodes and skin. Often patients with sarcoidosis will recover without any intervention within months or years. However, other patients will develop chronic sarcoidosis which will get worse over time, and interventions will be needed to manage symptoms. There is no cure so treatment is focused on managing the symptoms.

### Niclosamide

Niclosamide is a molecule having the structure:

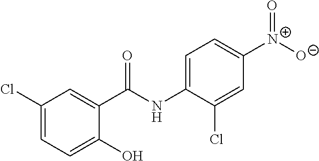 [SMILES: O=C(Nc1ccc([N+](=O)[O-])cc1Cl)c1cc(Cl)ccc1O]

Niclosamide was developed as an anthelminthic drug as it acts to inhibit anaerobic metabolism in the helminth or tape worm. It has since been noted that it may have other applications, including for the treatment of cancer where it may act as an inhibitor of mTORC1 signalling, an inhibitor of the IL-6-JAK-STAT3 pathway, an inhibitor of STAT3 signalling, an inhibitor of Wnt, notch, mTOR and NF-κB signalling, or an inhibitor of Wnt/β-catenin signalling, to name a few modes of action reported.

Reference herein to niclosamide includes salts thereof, preferably pharmaceutically acceptable salts of niclosamide. The salt may be an ethanolamine salt.

### IL-6 Inhibitors

An IL-6 inhibitor may block signal transduction by IL-6 or inhibit the biological activity of IL-6. The IL-6 inhibitor may inhibit binding between IL-6 and the IL-6 receptor. The IL-6 inhibitor may act directly or indirectly on IL-6. The IL-6 inhibitor may be an inhibitor of the IL-6 pathway, and may block signal transduction downstream of IL-6, either directly or indirectly. The IL-6 inhibitor may inhibit a transcription factor downstream IL-6/IL-6 receptor. The IL-6 inhibitor may inhibit STAT3.

The IL-6 inhibitor used in the invention may be niclosamide or a salt thereof.

The IL-6 inhibitor may be a small molecule or a biologic.

A biologic may include an antibody, or antigen binding fragment thereof, a nucleic acid, or a protein. A nucleic acid may be a DNA or RNA molecule. The nucleic acid may prevent or reduce expression of IL-6 or one of its binding partners, the nucleic acid may be an oligonucleotide, an siRNA or an shRNA.

The IL-6 inhibitor may be a monoclonal antibody, such as tocilizumab, sarilumab and siltuximab. Tocilizumab and sarilumab bind to the IL-6 receptor, while siltuximab binds to IL-6.

### STAT3 Inhibitors

STAT3 is a transcription factor encoded in humans by the STAT3 gene. It is active throughout the body and is involved in diverse pathways including wound healing, angiogenesis, immune pathways and cancer.

A STAT3 inhibitor may inhibit the biological activity of STAT3, for example by binding or altering the transcription activator activity of STAT3. The STAT3 inhibitor may inhibit or alter the interaction of STAT3 with one or more of its interaction partners, either directly or indirectly. The STAT3 inhibitor may have an inhibitory effect on transcription. The STAT3 inhibitor may reduce signal transduction downstream of IL-6. The STAT3 inhibitor may reduce biological activity induced by IL-6.

The STAT3 inhibitor used in the invention may be niclosamide or a salt thereof, or Stattic or a salt thereof, wherein Stattic has the structure:

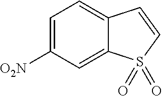 [SMILES: O=[N+]([O-])c1ccc2c(c1)S(=O)(=O)C=C2]

The STAT3 inhibitor may be a small molecule or a biologic. A biologic may include an antibody, or antigen binding fragment thereof, a nucleic acid, or a protein.

A nucleic acid may bind to STAT3 or one of its interacting partners. The nucleic acid may be a DNA or RNA molecule. The nucleic acid may prevent or reduce expression of STAT3 or one of its interacting partners, the nucleic acid may be an oligonucleotide, an siRNA or an shRNA.

### The IL-6/STAT3 Pathway Inhibitors

The IL-6/STAT3 pathway may provide a target for alternative treatments for granulomatous diseases. The IL-6/STAT3 pathway is outlined and discussed in Johnson et al. (Johnson, D., O'Keefe, R. & Grandis, J. Targeting the IL-6/JAK/STAT3 signalling axis in cancer. Rev Oncol 234-248 (2018). Nat Clin 15, https://doi.org/10.1038/nrclinonc.2018.8) and in FIG. 20 (JAK/STAT: IL-6 receptor signalling, 13 Jan. 2020, Cell Signaling technology; https://www.cellsignal.co.uk/pathways/jak-stat-il6-receptor-signaling).

An IL-6/STAT3 pathway inhibitor may block signal transduction from IL-6 or STAT3 or anywhere in the IL-6/STAT3 pathway. The IL-6/STAT3 pathway inhibitor may have an inhibitory effect against IL-6 or STAT3 directly or indirectly. The IL-6/STAT3 pathway inhibitor may have an inhibitory effect on other molecules in the pathway, for example Janus kinase (JAK).

The IL-6/STAT3 pathway inhibitor may be an IL-6 inhibitor and/or a STAT3 inhibitor as defined herein. The IL-6/STAT3 pathway inhibitor may be niclosamide or a salt thereof.

The IL-6/STAT3 pathway inhibitor may be a small molecule, or a biologic. A biologic may include an antibody, or antigen binding fragment thereof, a nucleic acid, or a protein.

A nucleic acid may bind to any molecule in the IL-6/STAT3 pathway or one of its interacting partners. The nucleic acid may be a DNA or RNA molecule. The nucleic acid may prevent or reduce expression of IL-6, STAT3, or one of their interacting partners in the IL-6/STAT3 pathway, the nucleic acid may be an oligonucleotide, an siRNA or an shRNA.

As used herein, reference to an antibody or antigen binding fragment thereof may refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, immunologically active antibody fragments (e.g., antibody fragments capable of binding to an epitope, e.g., Fab fragments, Fab′ fragments, F(ab′)2 fragments, Fv fragments, fragments containing a VL and/or VH Domain, or that contain 1, 2, or 3 of the complementary determining regions (CDRs) of such VL Domain (i.e., CDRL1, CDRL2, and/or CDRL3) or VH Domain (i.e., CDRH1, CDRH2, and/or CDRH3)) that specifically bind an antigen, etc., bi-functional or multi-functional antibodies, disulfide-linked bispecific Fvs (sdFv), intrabodies, and diabodies, and epitope binding fragments of any of the above. In particular, the term “antibody” is intended to encompass immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgAQ1 and IgA2) or subclass.

An antibody or antigen binding fragment thereof may include a bispecific antibody, comprising two different variable regions, each of which specifically bind different epitopes, either on the same or on different antigens.

The niclosamide or a salt thereof, or an inhibitor as described herein, may be provided in a composition, the composition may further comprise a carrier or excipient. The composition may be a pharmaceutical composition and may further comprise a pharmaceutically acceptable excipient or carrier. The carrier may be selected from the group comprising fillers, disintegrants, binders, humectants, extenders, dissolution re-tarders, absorption enhancers, wetting agents, adsorbents and/or lubricants. The composition may be a capsule, a tablet, a coated tablet, a nasal spray, an inhalant, a suppository, an ointment, a cream, an injection solution and/or an infusion solution. In an embodiment the niclosamide or a salt thereof, or the inhibitor as described herein, may be administered as a nasal spray or an inhalant. The niclosamide or a salt thereof, or the inhibitor as described herein, may be administered as a slow release formulation.

### Combination

The IL-6 inhibitor, the STAT3 inhibitor, the IL-6/STAT3 pathway inhibitor, or niclosamide or a salt thereof, composition or method of treatment described herein, may be combined with, or used in combination with, other known therapies for the treatment of a granulomatous disease. For example in combination with steroids and/or immunosuppressants.

As used herein, the term “combination” or “combined with” as it relates to therapy, refers to the use of more than one therapeutic. The use of the term does not restrict the order in which agents or pharmaceutical compositions are administered to a subject in need thereof.

### Methods of Treatment

A patient having a granulomatous disease may be treated by administering a therapeutically effective amount of niclosamide or a salt thereof, an IL-6 inhibitor, a STAT3 inhibitor, and/or an IL-6/STAT3 pathway inhibitor. Treatment may result in a reduction or removal of disease symptoms. Treatment may also prevent or reduce disease flares.

A variety of administration routes for niclosamide or a salt thereof, the IL-6 inhibitor, the STAT3 inhibitor, and/or the IL-6/STAT3 pathway inhibitor, or compositions of the invention are available. The particular mode selected will depend upon the particular inhibitor or composition selected, whether the administration is for prevention, or treatment of disease, the severity of the medical disorder being treated and dosage required for therapeutic efficacy. The methods of this invention may be practiced using any mode of administration that is medically acceptable, and produces effective levels of the active compounds without causing clinically unacceptable adverse effects. Such modes of administration include, but are not limited to, oral, buccal, sublingual, inhalation, mucosal, rectal, intranasal, topical, ocular, periocular, intraocular, transdermal, subcutaneous, intra-arterial, intravenous, intramuscular, parenteral, or infusion methodologies. In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferred modes of administration include oral, rectal, inhalation, intranasal or topical.

The niclosamide or a salt thereof, the IL-6 inhibitor, the STAT3 inhibitor, and/or the IL-6/STAT3 pathway inhibitor may be administered topically to the patient's skin. Alternatively, the niclosamide or a salt thereof, the IL-6 inhibitor, the STAT3 inhibitor, and/or the IL-6/STAT3 pathway inhibitor may be given via inhalation. Alternatively, the niclosamide or a salt thereof, the IL-6 inhibitor, the STAT3 inhibitor, and/or the IL-6/STAT3 pathway inhibitor may be administered intranasally. The niclosamide or a salt thereof, the IL-6 inhibitor, the STAT3 inhibitor, and/or the IL-6/STAT3 pathway inhibitor may alternatively be given systemically.

Niclosamide or a salt thereof, the IL-6 inhibitor, the STAT3 inhibitor, and/or the IL-6/STAT3 pathway inhibitor, is intended to be administered in a therapeutically effective amount. As used herein, the term “therapeutically effective amount” refers to the amount of the inhibitor or niclosamide or a salt thereof that is sufficient to provide patient benefit, i.e., prevention or amelioration of the condition to be treated or to prevent disease flares.

The precise dose of niclosamide or a salt thereof or the inhibitor as described herein to be administered depends on the route of administration, and the seriousness of the condition, and should be decided according to the judgment of the practitioner and each patient's circumstances and can be determined by standard clinical techniques. Effective doses may be extrapolated from dose-response curves derived from in vitro or animal model test systems. The particular dosage regimen, i.e., dose, timing and repetition, will thus depend on the particular individual and that individual's medical history, as well as the route of administration. The composition may be given once or twice a day. For oral administration, the composition may be administered in a dose of up to about 200 mg, about 300 mg, about 400 mg, about 500 mg, about 600 mg, about 700 mg, about 800 mg, about 900 mg, about 1 g about 1.2 g, about 1.4 g, about 1.6 g, about 1.8 g, or about 2 g, the dose may be between about 200 mg and about 2 g per dose, preferably between about 200 mg and about 1 g per dose. For nasal administration the composition may be administered in a dose of about 1 mg, about 1.1 mg, about 1.2 mg, about 1.3 mg, about 1.4 mg, about 1.5 mg, about 1.6 mg, about 1.7 mg, about 1.8 mg, about 1.9 mg or about 2.0 mg per dose. The dose may be between about 1 mg and about 2 mg, for example between about 1.2 mg and about 1.8 mg, or between about 1.2 mg and about 1.5 mg per dose.

Embodiments of the invention will now be described in more detail, by way of example only, with reference to the accompanying drawings.

### BRIEF DESCRIPTION OF THE FIGURES

FIG. 1—shows MPEg-cherry macrophage aggregation in zebrafish following PR3 stimulation. The figure shows a zebrafish model of GPA. Light, epifluorescence and confocal microscopy was used to confirm cell fusion at different time points. Tg (MPEg-cherry) zebrafish were anaesthetised at 24 hours post-fertilisation and injected with enzymatically active or inactive PR3 or albumin at 1 pg/ml into the yolk sac. Fish were imaged by lightsheet microscopy at 7 days post-fertilisation. In zebrafish (n=9/group), both enzymatically active and inactive PR3 injection was associated with a significant increase in cell fusion and aggregate volume (p<0.001) when compared to albumin injected controls.

FIG. 2—shows PR3 induces multinucleated giant cell (MGC) formation in GPA patients monocytes. PBMCs were isolated from healthy controls (n=10), GPA (n=10) and from microscopic polyangiitis (MPA) (n=10) patients. Monocytes were magnetically isolated. Cells were stimulated with either enzymatically active or inactive PR3 or control auto-antigen myeloperoxidase (MPO) at lug/ml or 10 μg/ml and incubated for 72 hours. (FIG. 2A). shows there was a dose response effect seen in GPA patients with increasing MGC formation at PR3 10 μg/ml, compared to MPA patients and healthy controls. MPO did not stimulate MGC formation. HNE was also tested (FIG. 2B), and showed a similar increase in MGC formation in GPA patients. Values plotted as median and 95% CI, *p<0.05; ** p<0.01; *** p<0.001, **** p<0.0001. The difference between conditions was determined by one-way and two-way ANOVA tests.

FIG. 3—shows that monocyte fusion is stimulated by PR3. Monocytes stimulated with PR3 were first imaged by Giemsa staining. Distinct cellular aggregates formed in the presence of PR3 (FIG. 3A). At higher magnification (FIG. 3B), a larger cellular aggregate is present showing dense nuclear staining and cytoplasmic fusion. (Scale bars 100 μm). (FIG. 3C, D) show 3D volume rendering of an aggregate including all acquired slices by confocal microscopy was undertaken using cell membrane (red), cytoskeletal (green) and nuclear staining (blue). Mid-slice (FIG. 3D) confirmed membrane fusion, cytoskeletal rearrangement and a single cellular structure with fusion of 3 monocytes, defining a MGC. Scanning electron microscopy highlighted monocyte fusion with representative images showing MGC size 48.8 μm (FIG. 3E) and 34.6 μm (FIG. 3F). Membrane fusion was measured by profile plot of fluorescence intensity of the three channels over the x/y diameter (FIG. 3G). There was prominent membrane staining encasing cytoskeleton and 3 nuclei.

FIG. 4—shows that PR3-induced MGC stimulates pro-inflammatory cytokines. Cytokines and chemokines were measured in MGC cell culture supernatants in unstimulated, PR3- or MPO-stimulated cells (FIG. 4A). IL-6 production increased in the presence of PR3 in GPA patients compared to MPA patients but was not significantly different to healthy controls (FIG. 4B). A reduction in IL-6 production was seen in GPA patients following stimulation with MPO. MCP-1 production (FIG. 4C) was also increased in GPA patients following stimulation with PR3 compared to MPA patients. MPO stimulation did not have any effect on differences in IL-6 or MCP-1 production. Values plotted as median and 95% CI, *p <0.05; ** p<0.01; *** p<0.001. Difference between multiple conditions was determined by two-way ANOVA.

FIG. 5—shows that MGC form in the presence of enzymatically active and inactive PR3. No statistical difference in fusion index was seen in GPA patients' cells when stimulated with enzymatically active or heat inactivated (Hi) PR3 (FIG. 5A). PAR2 agonism increased the fusion index in GPA patients and PAR2 antagonism significantly inhibited rates of MGC formation (FIG. 5B). The presence of PR3-ANCA or MPO-ANCA alone had no effect on the fusion index compared to control immunoglobulin (FIG. 5C), however, when cultured with PR3, there was a significant increase in MGC formation in the presence of PR3-ANCA and control immunoglobulin compared to MPO-ANCA. (FIG. 5D). PR3-ANCA GPA patients showed a significant increase in monocyte fusion compared to healthy controls and MPO-ANCA MPA patients, however a similar increase in MGC formation was seen in MPO-ANCA GPA patients. Values plotted as median and 95% CI, *p<0.05; ** p<0.01; *** p<0.001, **** p<0.0001. The difference between conditions determined by one-way and two-way ANOVA tests.

FIG. 6—shows that PR3, MAC-1 and PAR-2 expression is increased on GPA patients' monocytes. Following staining, proportions of classical (CD14 hi CD16lo), intermediate (CD14 hi CD16 hi) and non-classical (CD14lo CD16 hi) monocytes were calculated. There was a significant increase in intermediate monocytes in GPA patients compared to healthy controls and non-classical monocytes in GPA patients compared to MPA patients (FIG. 6A). PR3, MAC-1 and PAR-2 expression on CD14 positive GPA patients' monocytes was significantly increased compared to healthy controls and MPA patients (FIG. 6B). Values plotted as median and 95% CI, *p<0.05; ** p<0.01; *** p<0.001. The difference between conditions was determined by one-way and two-way ANOVA tests.

FIG. 7—shows that MGC formation is dependent on MAC-1, PAR-2 and IL-6. The effect of MAC-1 and IL-6 inhibition was tested in GPA patients by culturing monocytes with enzymatically active or inactive PR3 and measuring MGC formation in the presence or absence of anti-MAC-1 antibody, anti-IL-6 antibody or control immunoglobulin. Consistently, PR3 stimulated an increase in MGC formation as well as in the presence of control immunoglobulin with enzymatically active and inactive PR3 (FIG. 7A, B). Anti-MAC-1 antibody and anti-IL-6 antibody reduced MGC formation in both conditions and the effect of combined anti-MAC-1 antibody and anti-IL-6 antibody also significant reduced MGC formation. Values plotted as median and 95% CI, *p<0.05; ** p<0.01; *** p<0.001. The difference between conditions was determined by one-way ANOVA tests.

FIG. 8—shows that enzymatically-active and -inactive PR3 stimulates macrophage aggregation in zebrafish and is inhibited by niclosamide. MPEg-cherry zebrafish (n=10 per group) were injected with human PR3 or albumin (intact or following heat inactivation) and imaged by lightsheet microscopy. Aggregate volumes were calculated by 3D rendering of images following application of automated thresholds. Colour scale is used to demonstrate minimal and maximal aggregate volumes. Albumin with or without heat inactivation (FIG. 8A, B) showed minimal macrophage aggregation compared to enzymatically active and inactive PR3 at 5 pg/ml (FIG. 8C, D). Scale bar 100 μm. There was a significant increase in number and volume of macrophage aggregates in MPeg-cherry zebrafish injected with enzymatically active or inactive PR3 compared to albumin (FIG. 8E). niclosamide was administered to zebrafish following injection with PR3. Compared to PR3 alone (FIG. 8F, G), there was a reduction in aggregate volume and number in zebrafish injected with 0.3 μmol/ml, 0.15 μmol/ml and 0.05 μmol/ml niclosamide. Values plotted as median and 95% CI, *p<0.05; ** p<0.01; *** p<0.001. The difference between conditions was determined by two-way ANOVA.

FIG. 9—shows the effect of PR3 and albumin injection at different doses in zebrafish with or without niclosamide administration to the fish tank water.

FIG. 10—shows that ConA conditioned media and PR3 stimulates MGC formation. Whole blood PBMCs were stimulated with ConA at 16 μg/ml in complete medium for 72 hours and conditioned media used to stimulate CD14 positive monocytes isolated from PBMCs at 50% concentration in complete medium. Cells were stained with modified Giemsa and microscopy performed and MGC fusion index calculated, using the threshold defined by scanning electron microscopy of greater than 37 μm. Unstimulated cells (FIG. 10A) show minimal cell fusion (4.95 (2.1-6)) compared to stimulated cells (FIG. 10B) (24.7 (19.5-32.9)). Values represented as median and 95% confidence intervals, n=6 (FIG. 10C). Significant difference between groups determined by one-way ANOVA test. ** p<0.01. Following this, purified isolated monocytes from healthy controls (n=4) were stimulated with PR3 10 μg/ml and fusion index measured at days 1, 3 and 7 (FIG. 10D). There was no evidence of fusion at day 1 (median fusion index 0.96; CI 0.12-3.62) but by day 3 (8.66; 5.35-10.82) and day 7 (9.86; 7.01-11.6) there was a significant increase in fusion index (p<0.001; one-way ANOVA test). No difference in fusion index was observed between days 3 and 7 and so a 3-day time point was used for all further experiments.

FIG. 11—shows that heat inactivation of PR3 enzymatic activity. PR3 enzymatic activity was measured using the chromogenic substrate of N-methoxysuccinyl-Ala-Ala-Pro-Val p-nitroanilide and quantified by OD values at 405 nm. Following heat inactivation by incubating PR3 at 100° C. for 15 minutes, there is loss of enzymatic activity at varying concentrations. Results are given as mean values, n=3.

FIG. 12—illustrates representative FACS gating strategy for monocyte staining. Whole blood was stained with CD14, CD16, PR3, MACI and PAR2 fluorescent antibodies. Monocytes were first gated by FSC and SSC. Fluorescence minus one (FMO) control was tested for each fluorochrome. Representative FACS plots shown for one sample.

FIG. 13—shows that granuloma like structures form following PR3 stimulation of PBMC. Whole peripheral blood mononuclear cells (PBMC) isolated from three different GPA patients were stimulated with PR3 (10 μg/ml) and cultured for different time points up to three days. The whole cell population was divided into two and labelled with either PKH26 (red) or CFSE (green), before being re-combined. This dual cell labelling allowed visualisation of the interaction between cells and the extent of fusion to be determined with CFSE and PKH26 labelled cells producing prominent yellow/brown fluorescent staining throughout the structure representing cellular fusion. Evidence of cell co-localisation was seen by 4 hours (FIG. 13A, magnification ×200) but a clear increase in cell aggregation and fusion only occurred by 24 hours. After 72 hours (FIG. 13B, magnification ×400) more extensive cell fusion was readily visible. Scale bar 200 μm.

FIG. 14—shows that granuloma are composed of fused CD14+giant cells and CD3+ T cell aggregates. Composite images by confocal microscopy PBMCs stimulated with PR3 for 72 hours of two different stacks at the top (FIG. 14A) 26/73 and middle (FIG. 14D) 45/73 of a 73-slice Z stack are shown, as well as addition monochrome images; CD3 (green) (FIG. 14B, E) and CD14 (red) (FIG. 14C, F). At the top of the Z stack, CD3 staining is more intense demonstrating a predominance of T cells whereas the opposite is seen in the middle of the Z stack with prominent monocyte staining. Immunofluorescent image of PBMCs cultured with PR3 at a concentration of 10 μg/ml (FIG. 14G, H). Cells were stained with membrane stain (WGA (Alexa Fluor 594)). Different shapes and sizes of cellular aggregates are present. Membrane staining is red in colour and increased stain intensity is demonstrated around the circumference of each cell cluster with minimal staining within, suggesting the presence of a shared membrane. The nuclei are observed by the silhouette appearance with the centre of the fused cells. Scale bar represents 100 μm.

FIG. 15—shows MGC formation by monocyte subsets. Monocytes were positively selected from PBMCs by CD14 cell labelling and flow cytometric sorting. Cells were then sorted based on MACI and PAR2 expression. (FIG. 15A) Unstained cells showed no MACI or PAR2 expression. (FIG. 15B) Stained cells confirmed double positive MACI and PAR2 expression. (FIG. 15C) Sorted cells were then stimulated with PR3 and fusion index measured, as previously, confirming increased monocyte fusion in monocytes expressing both MACI and PAR2 (p<0.05, Mann Whitney U test).

FIG. 16—shows binding of PR3 to Mac-1 using a FACS-based proteinase 3 (PR3)-binding assay. HEK-293T cells were transfected with the expression constructs as indicated. HEK-293T transfectants were collected, washed, and incubated in blocking buffer (1% BSA, 5% NGS/PBS) for 1 h at 4° C. before incubating with commercially available purified human PR3 (5 mg/ml) (Athens, USA) diluted in blocking buffer for 1 h at 4° C. Cells were subjected to extensive washes in cold PBS, and then incubated with the anti-PR3 mAb (5 mg/ml) (clone PR3-G2, Thermo Fisher Scientific) for 1 h at 4° C. Cells were extensively washed and then incubated with fluorescence-labelled secondary antibody for 1 h at 4° C. Finally, cells were washed three times in cold PBS and subjected to analysis by FACScan flow cytometer (BD Biosciences).

FIG. 17—shows imaging macrophage fusion. Representative images of zebrafish injected in the yolk sac with PR3 5 pg/mL and imaged at day 5 by lightsheet microscopy. (FIG. 17A) Binary maximum projection micrograph showing fusion in Mpeg-cherry labelled macrophages (>346 μm²) outlined in yellow. (FIG. 17B) Maximum projection image showing macrophage fusion (yellow) found using quantitative particle analysis. (FIG. 17C) Single Z-stack slice (slice 32 of 99) demonstrating macrophage fusion at a voxel depth of ~4.9 μm.

FIG. 18—details patient demographics and clinical characteristics

FIG. 19—shows that THP1 monocytes stimulated with PR3 and treated with the STAT3 inhibitor Stattic demonstrate a lower giant cell formation index than the control group or cells that are not treated with Stattic.

FIG. 20—shows the IL-6/STAT3 pathway (JAK/STAT: IL-6 receptor signalling, 13 Jan. 2020, Cell Signaling technology; https://www.cellsignal.co.uk/pathways/jak-stat-il6-receptor-signaling).

### EXAMPLES

The following examples show that niclosamide, IL-6 inhibitors, STAT3 inhibitors, and IL-6/STAT3 pathway inhibitors (which may include niclosamide, IL-6 inhibitors, and/or STAT3 inhibitors), may be used to treat granulomatous diseases.

### Example 1—In Vitro and In Vivo Models Show that Granuloma Formation in GPA May be Caused by Excessive PR3 Expression

Granulomas are organised aggregates of inflammatory cells, that can serve to isolate and contain certain infectious antigens or chronic irritants that are not readily eliminated through conventional immune responses. Although commonly associated with infectious diseases, a significant number of autoimmune, autoinflammatory or immunodeficient conditions result in granuloma formation in various body sites and may also serve as a final protective mechanism for the host, in many cases to unknown antigens. The formation of granuloma may be due to unknown infectious antigens (as has been suggested for sarcoidosis) or better known self-antigens, some of which are already known targets in autoimmunity (e.g. PR3 in ANCA vasculitis) or remain unknown (e.g. in Crohns disease). At the heart of the granuloma are specialised macrophages that fuse and form into multi-nucleate giant cells (GGCs), with augmented phagocytic and bactericidal capabilities. The pathogenesis of granuloma in the context of many autoimmune diseases, and particularly in ANCA-associated vasculitis, remains poorly understood, but is critically important, as persistent granuloma contribute to tissue damage and significant morbidity, resulting in exposure of patients to prolonged immunosuppressive medications, which leads to further adverse effects.

The hypothesis that persistent proteinase 3 (PR3), found in greater abundance in GPA patients, and known to frustrate macrophage mediated clearance, may be critical in promoting granuloma formation was tested by the inventors. The data presented here shows that in vitro PR3 and not MPO promotes greatest monocyte cell fusion and MGC formation, which is associated with elevated levels of secreted IL-6 and MCP-1. Elevated levels of some of the cytokines associated with granuloma formation in the context of mycobacterial infections (such as TNF-α, or IL-17) was not observed. Work from clinical cohorts has also shown significant differences in circulating IL-6 between GPA and MPA patients, supporting the findings that IL-6 is a key cytokine in mediating MGC and granuloma formation in GPA patients.

ANCA associated vasculitis GPA is characterised by ANCA reactivity towards PR3, an abundance of PR3 in granulomatous lesions and increased neutrophil membrane PR3 expression, while augmented PR3 on apoptotic cells inhibits their phagocytosis by macrophages. Therefore, in light of the discovery that IL-6 is a key cytokine mediating granuloma formation, IL-6 pathway inhibitors may represent a new avenue for treatment of GPA.

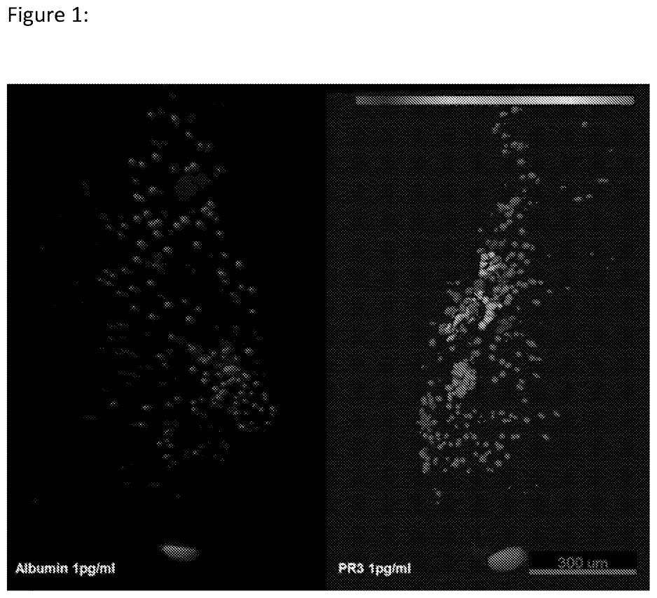

Monocyte aggregation and giant cell formation occurred following stimulation with both enzymatically active and inactive PR3 with a greater number (p<0.01) and size of aggregates (p<0.001) in GPA patients compared to MPA patients and healthy controls. Typical granuloma cellular organisation was observed following PBMC fusion with a greater number (p<0.001) and size (p<0.001) of aggregates seen in GPA patients. There was no significant difference between enzymatically active or inactive PR3. No effect was seen with MPO. Supernatant profiling implicated specific roles for pro-inflammatory cytokines and chemokines. In zebrafish (n=9/group), both enzymatically active and inactive PR3 was associated a significant increase in cell fusion and aggregate volume (p<0.001) when compared to albumin injected controls (see FIG. 1).

### Multinucleate Giant Cell Formation in GPA Patients is Promoted by PR3

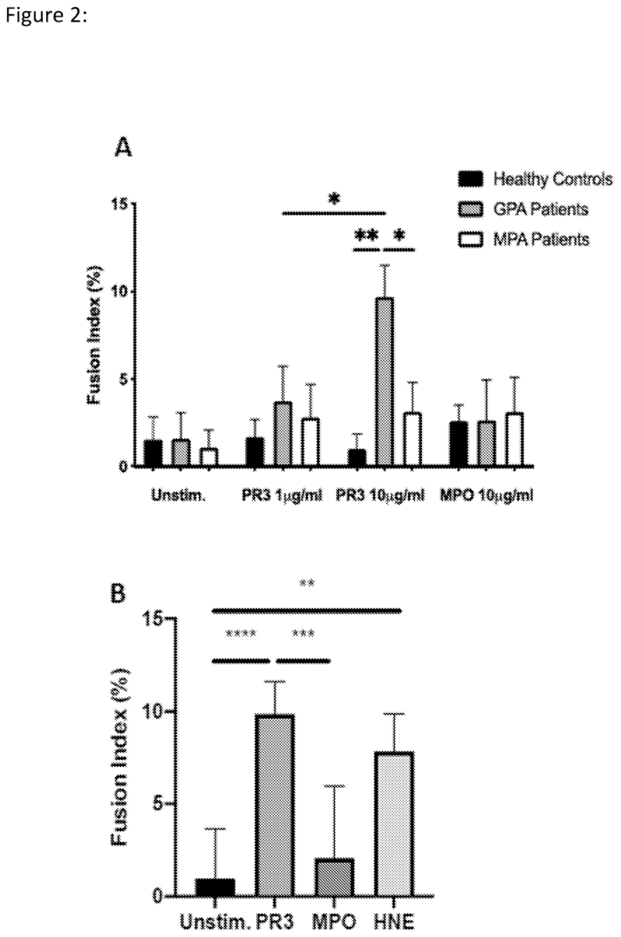

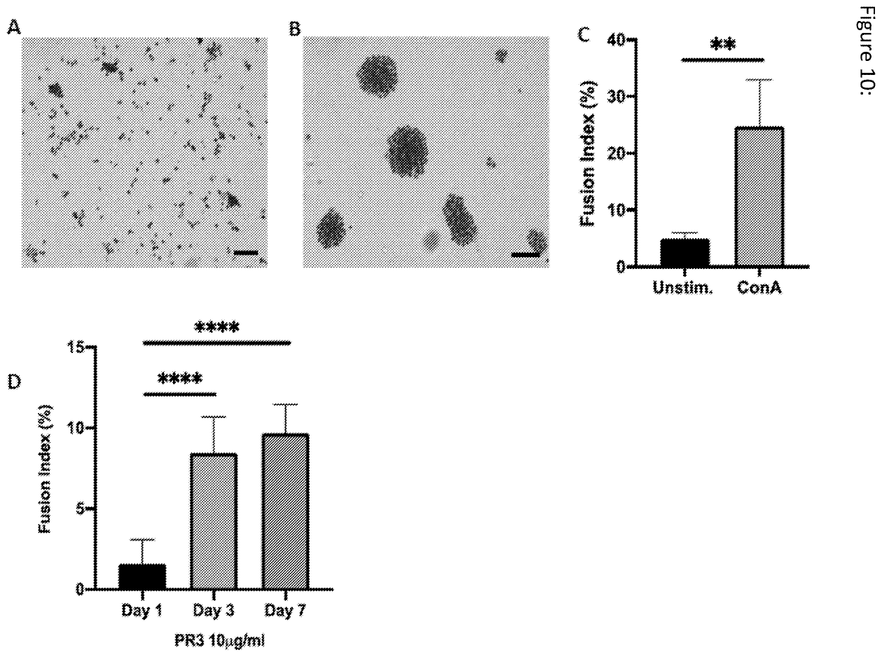

Experiments were undertaken to demonstrate that MGC cell formation in GPA patients is promoted by PR3. Initially, to demonstrate that MCG formation could be observed in vivo in blood samples, cells were stimulated with conA, which is well known to stimulate MGC formation. Peripheral blood mononuclear cells were used to isolate monocytes which were confirmed to be consistently >90% pure, based on CD14 staining (data not shown). Concanavalin (ConA) conditioned media is an established stimulus in models of in vitro MGC formation (Most J, Spotl L, Mayr G, Gasser A, Sarti A, Dierich M P. Formation of multinucleated giant cells in vitro is dependent on the stage of monocyte to macrophage maturation. Blood. 1997; 89 (2): 662-71.) and was used initially to validate experimental conditions. Quantification of cell fusion was based on unbiased Image J analysis. To confirm fusion rather than cell coalescence, cultured cells were washed prior to fixing in methanol and stained with Giemsa solution. There was a significant increase in MGC formation, assessed by the fusion index, in GPA patients stimulated with 50% ConA conditioned media (unstimulated cells median 4.95; confidence interval (CI) 2.1-6 vs. stimulated cells 24.7 (19.5-32.9) ((n=6) p<0.01; Mann-Whitney U test) and typical morphological appearances of substantial, well-defined cellular aggregation and monocyte fusion (see FIG. 10). Having demonstrated that MGC formation could be observed, the inventors then demonstrated that this could be promoted by PR3. To do this, monocytes were stimulated with PR3 10 μg/ml and fusion index measured at days 1, 3 and 7 (see FIG. 2A which shows the results after 3 days). There was no evidence of fusion at day 1 (median 0.96; CI 0.12-3.62) but by day 3 (8.66; 5.35-10.82) and day 7 (9.86; 7.01-11.6) there was significant increase in fusion index (n=4, p<0.001; one-way ANOVA test) as clearly demonstrated in FIG. 2A. No difference in fusion index was observed between days 3 and 7 and so a 72 hour time point was used for all future experiments.

PR3 concentrations lug/ml and 10 μg/ml were then tested in GPA patients, MPA patients and healthy controls (see FIG. 2B) as well as MPO 10 μg/ml, the predominant autoantigen in MPA. There was an increase in MGC formation in GPA patients when PR3 concentration increased from lug/ml (median 3.17; CI 2.35-5.68) to 10 μg/ml (9.86; 7.01-11.6) with no such effect seen in MPA patients (2.23; 1.22-4.8 to 2.8; 1.73-4.79) or healthy controls (1.37; 0.62-2.88 to 1.17; 0.14-1.33) (n=10 in all groups) (p<0.05; two way ANOVA). MGC formation in GPA patients was also significantly increased in response to PR3 stimulation at 10 μg/ml (9.86; 7.01-11.6) compared to healthy controls (1.17; 0.14-1.33) (p<0.01; two-way ANOVA) and MPA patients (2.8; 1.73-4.79) (p<0.05; two-way ANOVA). There was minimal MGC formation when cells were unstimulated, but importantly, no significant MGC formation was present in any group stimulated with MPO. In addition, the effect of another neutrophil serine protease, human neutrophil elastase (HNE), was tested on GPA patients' monocytes (FIG. 1C) and at comparable doses used for PR3, HNE also promoted MGC formation (9.3; 5.71-11.11 vs PR3 9.86; 7.01-11.6) (n=6) (p<0.01; two-way ANOVA test). These results clearly demonstrate a role for PR3 in MGC formation in GPA patients.

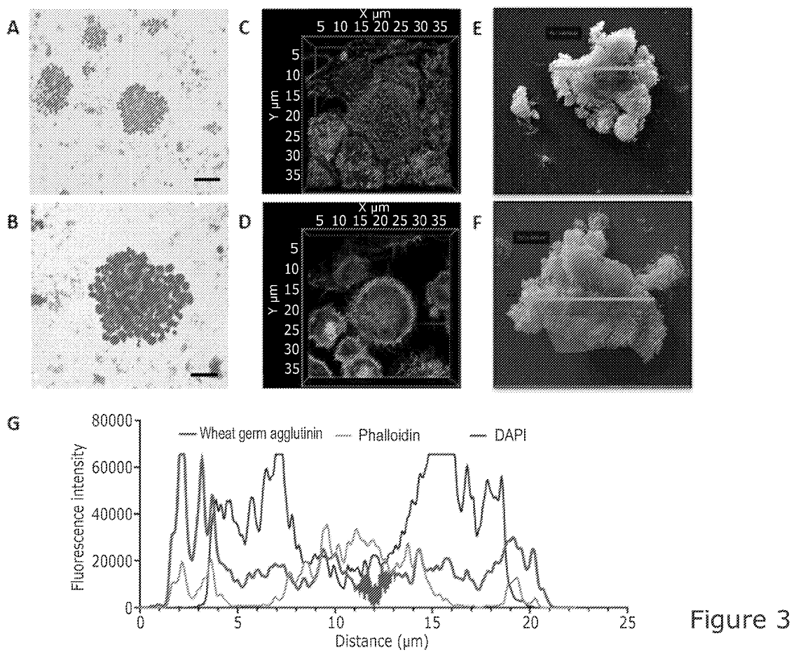

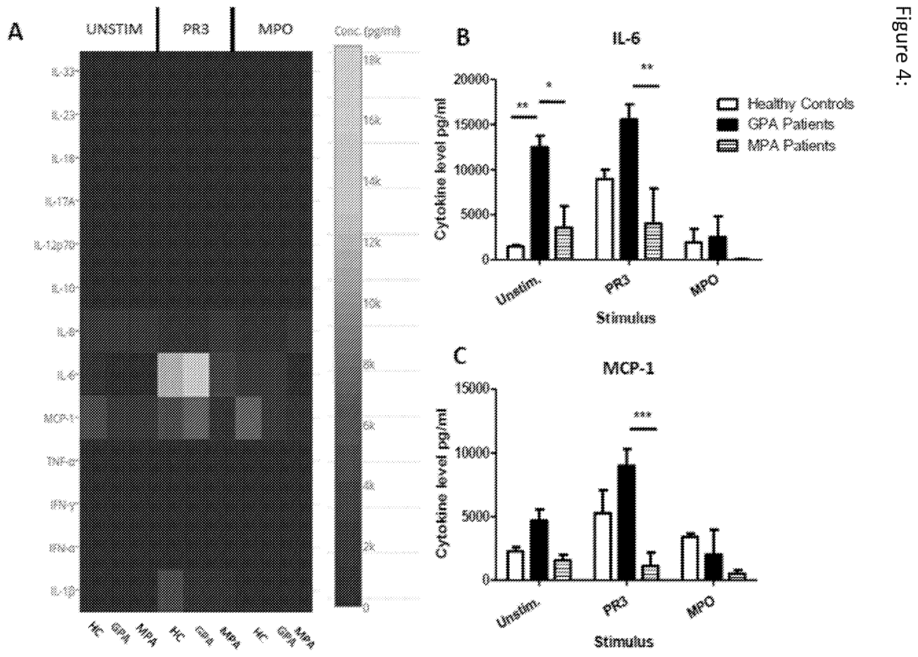

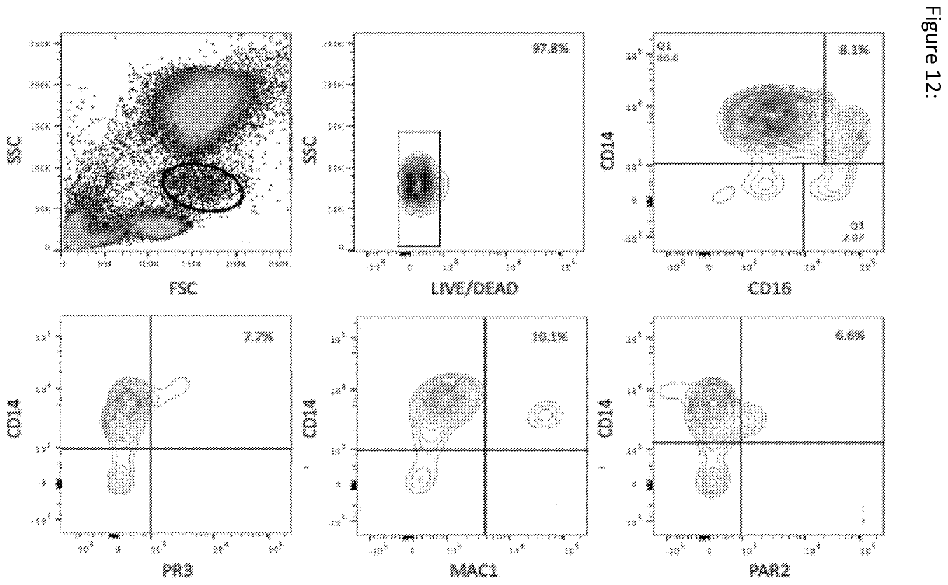

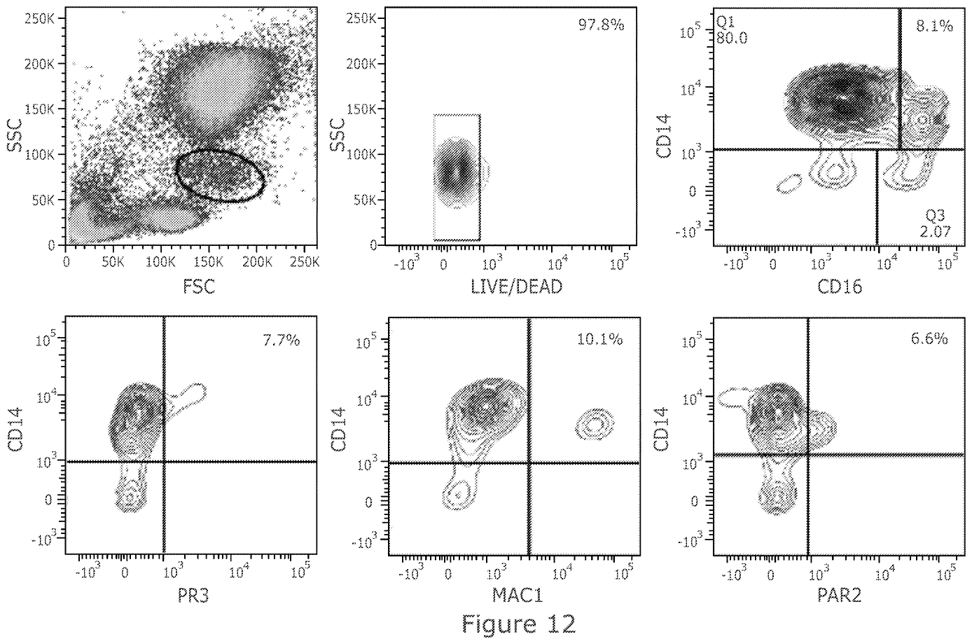

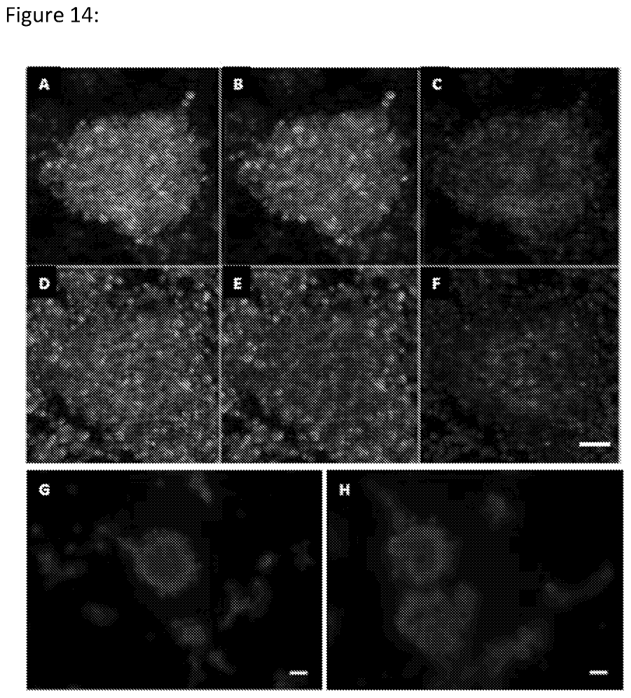

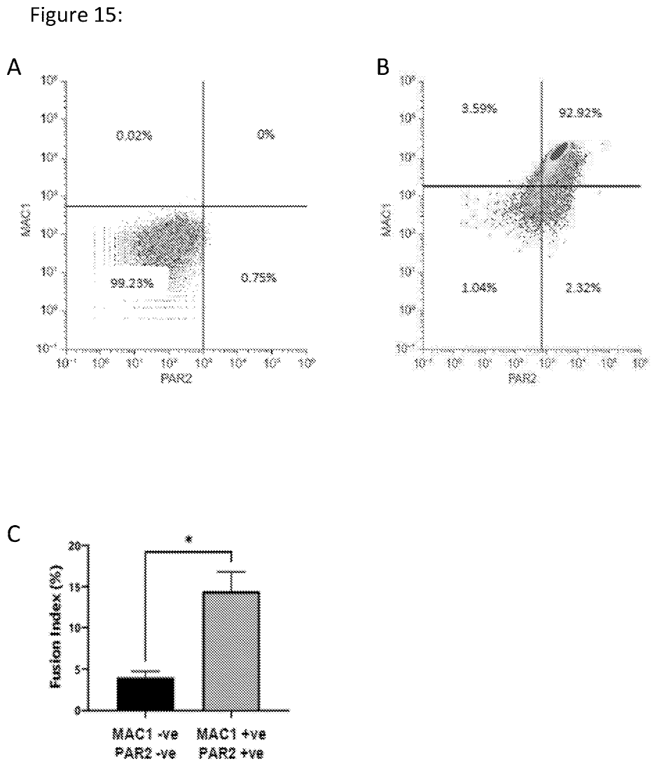

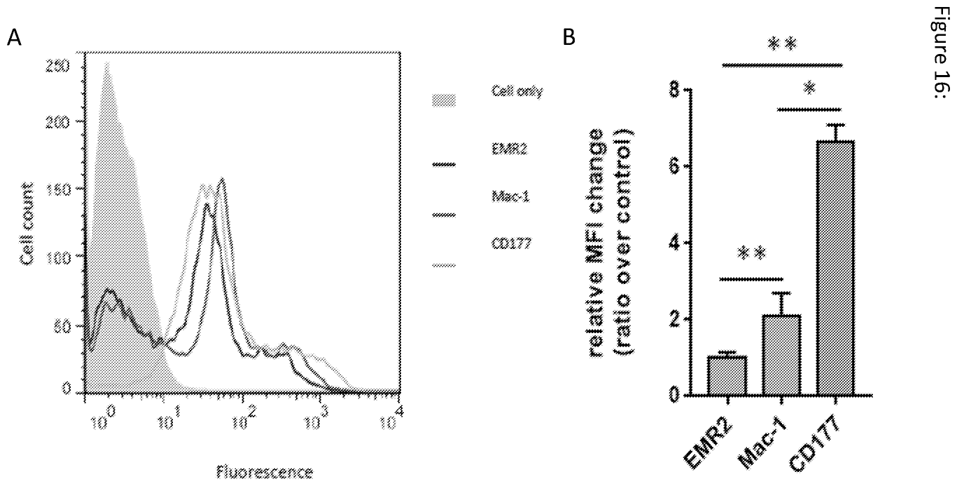

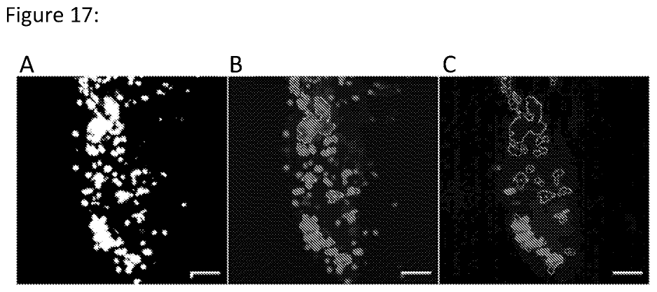

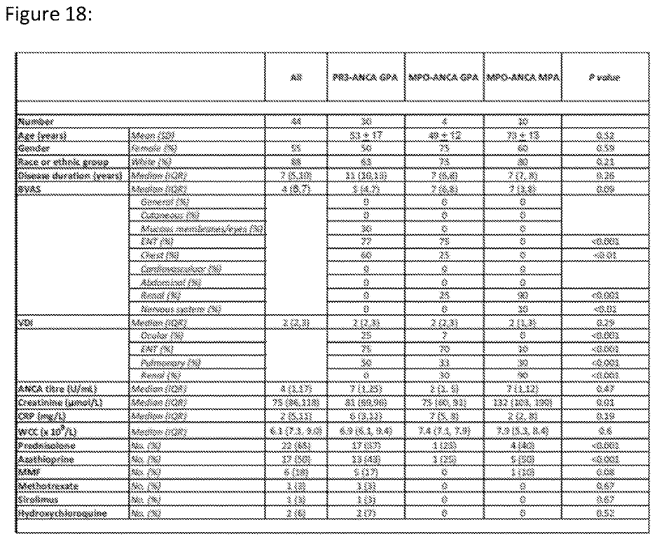

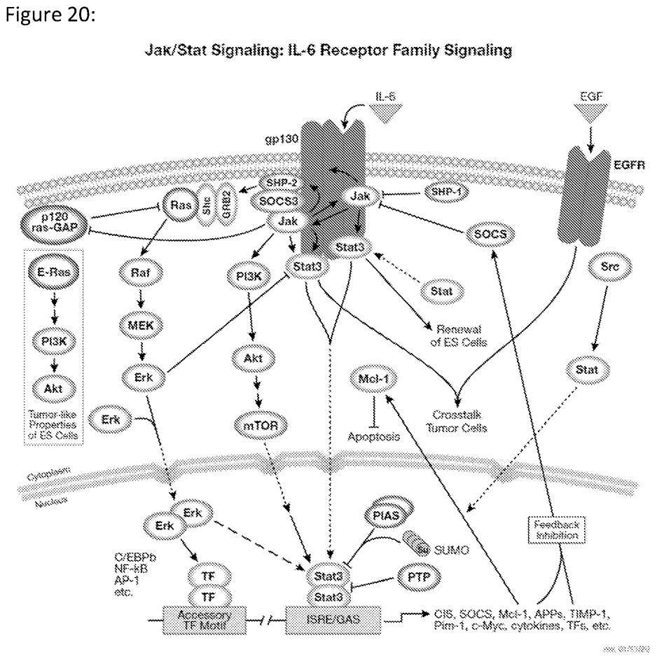

MGC formation was confirmed anatomically by Giemsa staining as well as by combined cell membrane (using wheat germ agglutinin (WGA)), actin (using phalloidin) and nuclear staining (using DAPI) (n=3) and confocal microscopy. Additionally, scanning electron microscopy (n=3) to evaluate the surface topography of fused cells was carried out (see FIGS. 3A-F). At varying magnifications, Giemsa imaging (see FIGS. 3A and 3B) demonstrated classical morphological features of MGC with a large cellular aggregate showing dense nuclear staining and cytoplasmic fusion. Confocal microscopy with 3D rendering demonstrated prominent outer membrane staining (see FIG. 3C) with fused cells contained within a single cellular structure (see FIG. 3D). Orientation of membrane, cytoskeletal and nuclear staining was also measured by profile plot of 3 channels fluorescence intensity over the x/y diameter (see FIG. 3G). Finally, MGC diameters were measured by scanning electron microscopy in 3 examples. Representative images show a range in size from 48.8 μm (see FIG. 3E) to 34.6 μm (see FIG. 3F).

### IL-6 is Upregulated in PR-3 Induced MGC Formation

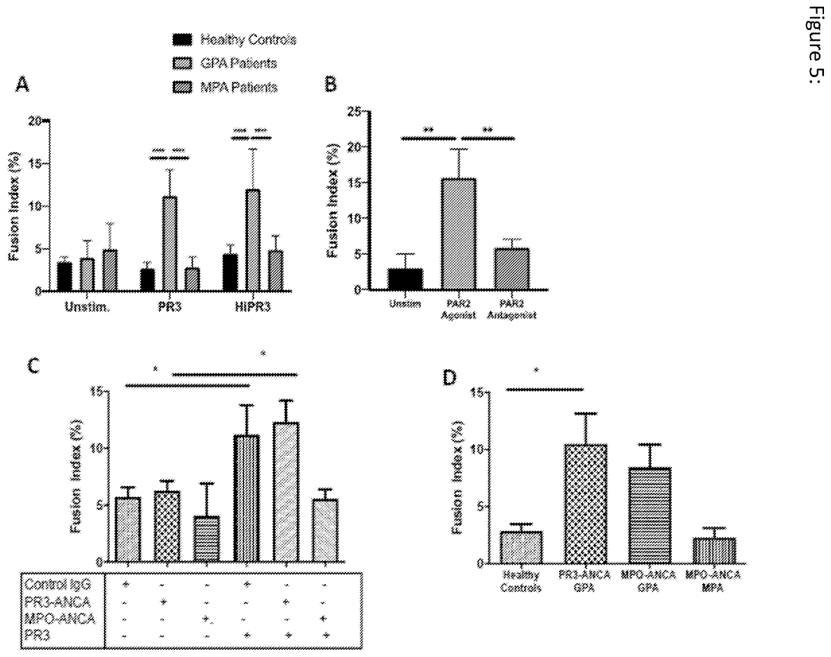

PR3-induced MGC formation is associated with pro-inflammatory cytokine production Cytokine arrays were used to quantify the cytokines and chemokines in unstimulated, PR3- or MPO-stimulated MGC culture supernatants using HC, GPA or MPA patients' monocytes (n=4 in all groups) (see FIG. 4A). IL-6 was produced more in unstimulated GPA patients' monocytes than in HC or in MPA patients (unstimulated GPA median IL-6 12379 μg/ml (IQR 9999-14992), HC 1419 μg/ml (1231-1806), MPA 2020 pg/ml (23-8637), GPA vs HC p<0.01, GPA vs MPA p<0.05; two-way ANOVA) (see FIG. 4B). Following PR3 stimulation, GPA patients' monocytes augmented IL-6 significantly more than MPA patients, but this was not significantly different to HC (GPA, median IL-6 15952 μg/ml (IQR 12281-18398), HC 8956 (6916-10946), MPA 114 (31-11850); GPA vs MPA p<0.01; two-way ANOVA) (see FIG. 4B). Following MPO stimulation there was a non-statistical reduction in IL-6 production, most marked in GPA patients' cells. For MCP-1, unstimulated GPA cells produced most MCP-1 but this was not statistically different to HC or MPA patients' cells (GPA median MCP-1 9161 (6379-11422), HC 4362 (2486-8928), MPA 48 (37-3252), however, following PR3 stimulation, GPA cells incremented MCP-1 production the most (GPA median MCP-1 4488 pg/ml (IQR 3124-6445), HC 2045 (1809-2960), and MPA 1593 (663-2398) GPA vs MPA p<0.001, GPA vs HC ns; two-way ANOVA) (see FIG. 4C); No significant changes were seen following MPO stimulation between the three groups. In addition, no significant differences were found in levels TNF-α, IFN-α, IFN-γ, IL-10, IL-8, IL-17, IL-12, IL-18, IL-23, or IL-33 with or without stimulation.

The data presented herein clearly demonstrates that PR3 can be used to promote MGC formation, and this provides a model system in which to look for inhibitors of MGC formation and hence therapies for treating granulomatous diseases.

### Modulation of MGC Formation in GPA Patients' Cells

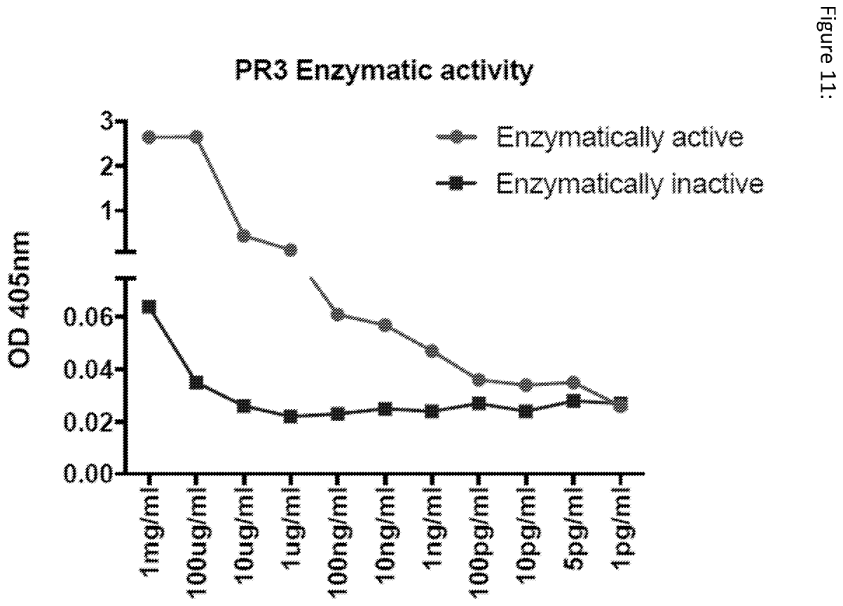

Experiments were conducted to shows that PR3 can be used to modulate MGC formation in GPA patients cells. PR3 enzymatic activity can mediate binding and cleavage of protease activated receptor-2 (PAR-2), activating monocytes which can be inhibited by alpha-1 antitrypsin (A1AT). In addition, non-enzymatic antigenic activity, may be mediated through binding other surface receptors such as MAC-1 or calreticulin. Isolated monocytes from GPA patients were cultured with enzymatically active PR3 or enzymatically inactive PR3, confirmed by enzymatic assay (FIG. 11), for 72 hours and fusion index calculated as before. Both enzymatically active and heat inactivated PR3 produced significant MGC formation in GPA patients' cells compared to unstimulated cells ((n=8) p<0.01; two-way ANOVA) (see FIG. 5A-B). There was no difference in MGC formation between enzymatically active or inactive PR3 stimulation, suggesting that different pathways may be involved in mediating the effect.

MAC-1 and PAR-2 Expression is Increased on GPA Patients' Monocytes and, Along with IL-6 are Critical for MGC Formation.

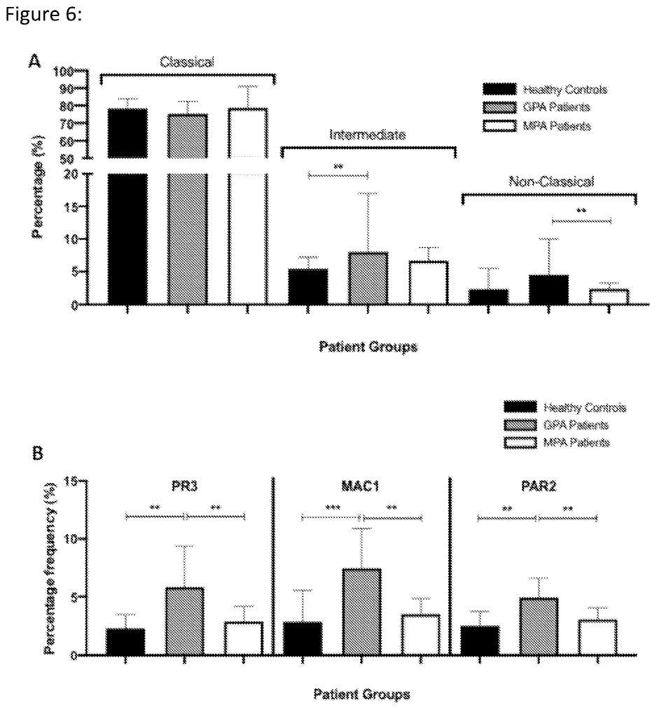

Since PR3 promotes MGC formation more readily in GPA rather than MPA patients, differences in monocyte populations and cell surface binding partners between GPA and MPA patients were tested. It was found that both enzymatically active and inactive PR3 may promote MGC formation, and since PR3 may bind cell surface Mac-1 (30), differences in cell surface expression of MAC-1 as well PAR-2 receptor were investigated. Classical, intermediate, and non-classical monocytes were identified by CD14 and CD16 staining (FIG. 5A-C). There was no difference in frequency of classical monocyte subsets between groups but an increase in intermediate monocytes in GPA patients compared to healthy controls ((n=6) p<0.01; one-way ANOVA) and non-classical monocytes in GPA patients compared to MPA patients ((n=6) p<0.01; one-way ANOVA) (FIG. 6A). Subsequently, percentage CD14 positive cells expressing PR3, MAC-1 and PAR-2 were measured ((n=6) in all groups) (FIG. 5D-F). Monocyte cell surface PR3 expression was significantly increased in GPA patients (median 5.78% (IQR 4.74-6.68)) compared to healthy controls (3.47% (IQR 3.15-3.47)) and MPA patients (3.44% (IQR 3.31-4.13)) (both p<0.01; one-way ANOVA). Monocyte MAC-1 expression was significantly increased in GPA patients (6.76% (IQR 6.27-7.74)) compared to healthy controls (3.55% (2.89-3.95)) (p<0.001; one-way ANOVA) and MPA patients (3.73% (IQR 3.3-3.87)) (p<0.01; one-way ANOVA). Similarly, PAR-2 cell surface expression on monocytes was significantly increased in GPA patients (4.79% (IQR 4.64-5.09)) compared to healthy controls (2.98% (IQR 2.05-3.71)) and MPA patients (3.21% (IQR 2.92-3.77)) (both p<0.01; one-way ANOVA) (FIG. 6B).

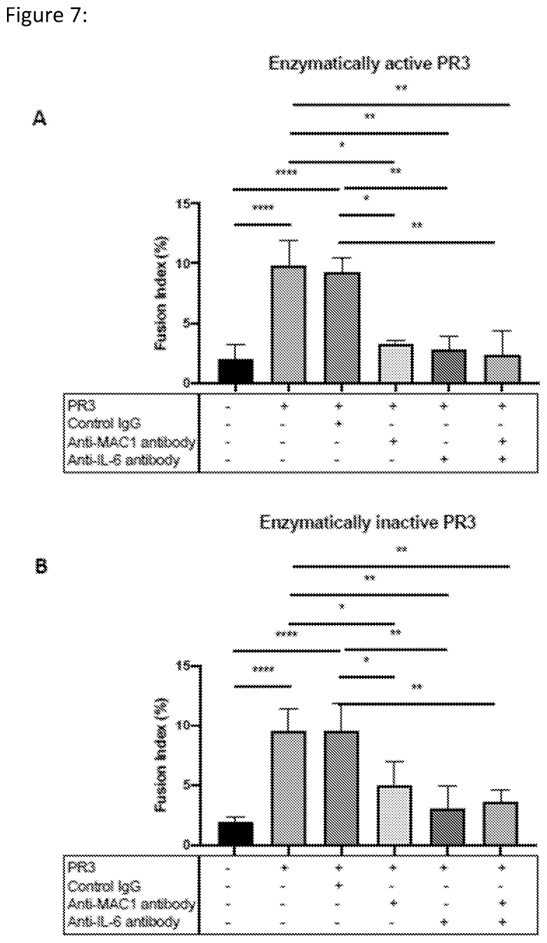

As the importance of blocking the enzymatic effect of PAR-2 was already identified, the effect of MAC-1 non-enzymatic PR3 binding partner was tested using anti-MAC-1 antibody in the presence of enzymatically active or inactive PR3 in GPA patients (n=4). IL-6 was also identified as a critical mediator of MGC formation in vitro and so inhibition of IL-6 with anti-IL-6 antibody was also tested in the presence or absence of anti-MAC-1 antibody (see FIG. 7A, B). Enzymatically active and inactive PR3 was again associated with a significant increase in MGC formation using GPA patients' cells compared to unstimulated cells (p<0.001; one-way ANOVA) and the presence of control immunoglobulin had no effect (p<0.001; one-way ANOVA). However, the presence of anti-MAC-1 antibody significantly reduced MGC formation in enzymatically active and inactive PR3 stimulated monocytes (p<0.05; one-way ANOVA), as did anti-IL-6 antibody (p<0.01; one-way ANOVA). The combined effect of anti-MAC-1 and anti-IL-6 antibodies was a similar reduction in MGC formation, again in the presence of either enzymatically active or inactive PR3, suggesting that both PR3 binding both PAR-2 and MAC-1 can mediate MCG formation.

### Granuloma Like Structures Form Following PR3 Stimulation of PBMC

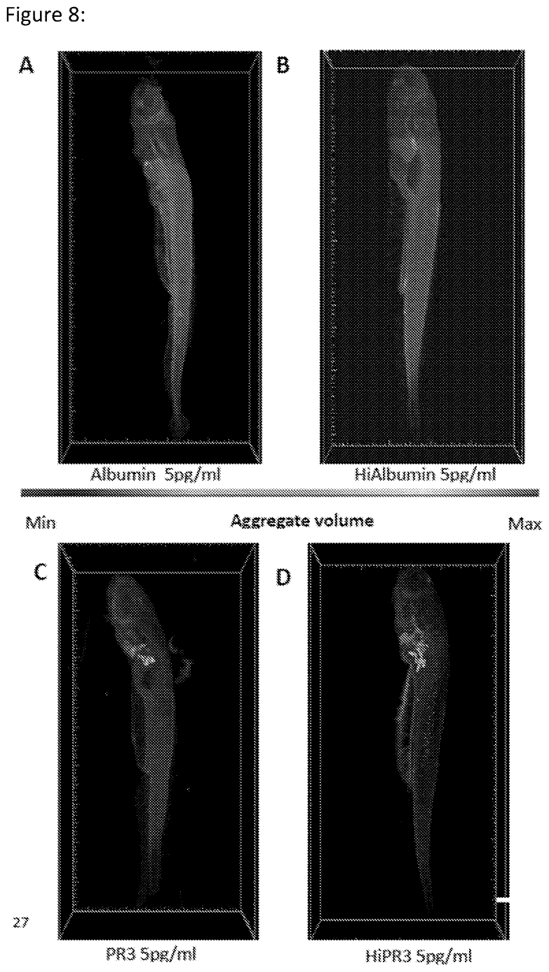

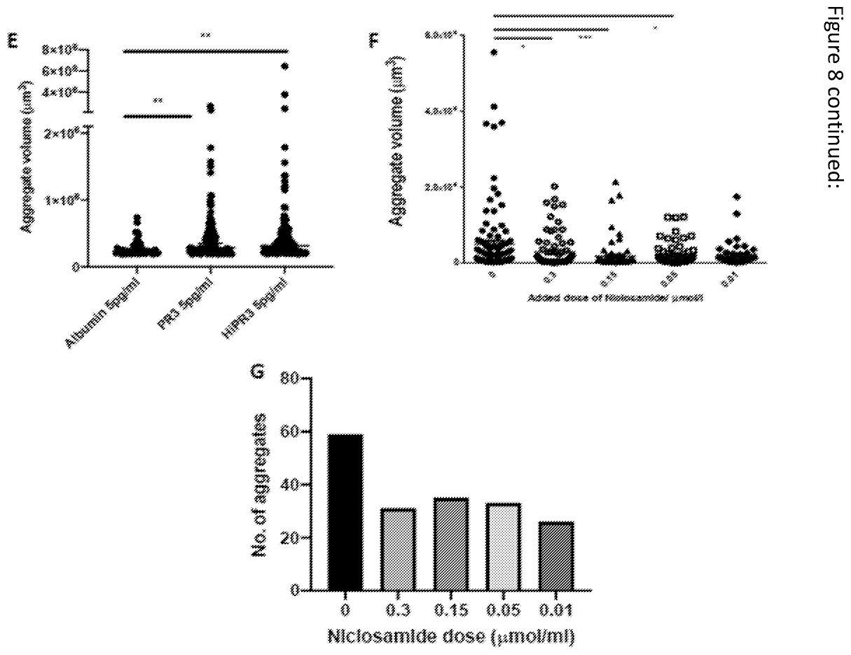

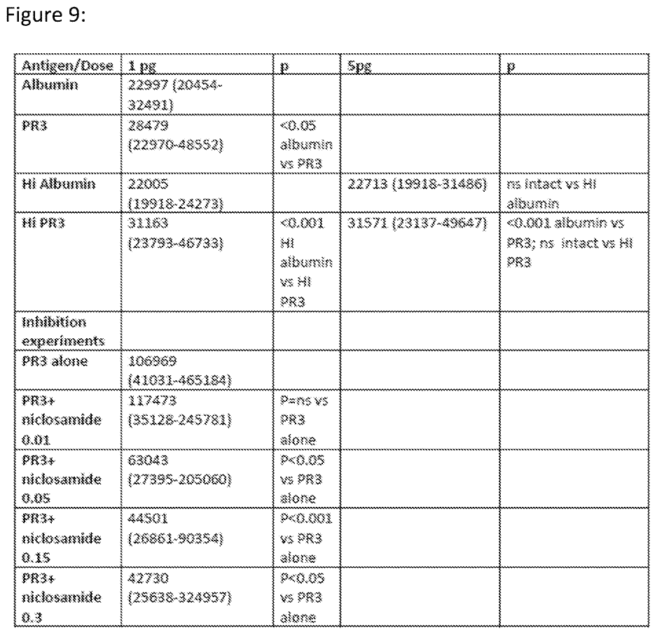

Experiments were conducted to show that granuloma like structures form after cells are stimulated with PR3. Whole peripheral blood mononuclear cells (PBMC) isolated from three different GPA patients were stimulated with PR3 (10 μg/ml) and cultured for different time points up to three days. The whole cell population was divided into two and labelled with either PKH26 or CFSE, before being re-combined. This dual cell labelling allowed visualisation of the interaction between cells and the extent of fusion to be determined. Evidence of cell co-localisation was seen by 4 hours (FIG. 8A, B) but a clear increase in cell aggregation and fusion only occurred by 24 hours. After 72 hours more extensive cell fusion was readily visible. Following staining for CD14 and CD3, as well as nuclei (using DAPI) and imaging by confocal microscopy, an inner core of CD14 positive cells was demonstrated, surrounded by an outer core of CD3 positive T cells (FIG. 9A-F). In addition, specific fusion of cells was clearly identified by single cell membrane staining (using WGA) that excluded features of cellular coalescence and aggregation, but rather confirmed PBMC membrane fusion (FIGS. 9G, H).

### Example 2—an In Vivo Model of Granulomatous Disease in Zebrafish was Used to Demonstrate that Niclosamide May be Used to Reduce Granuloma Formation Via Inhibition of the IL-6 STAT3 Pathway

An in vivo model of granulomatous disease in zebrafish was developed by administering PR3 to the fish. Niclosamide was administered to the fish via the fish tank water and the model shows that niclosamide has the capacity to reduce IL-6 levels and to significantly inhibit granuloma formation.

The injection of intact or HI PR3 into the zebrafish was repeated in the presence or absence of the IL-6/STAT3 inhibitor niclosamide, which was added to the fish water at different concentrations (0.01-0.3 μmol/l) (FIG. 11B, C). Since no difference between PR3 and HI PR3 was noted with respect MGC formation, all the fish injected with PR3 alone with those injected with differing doses of niclosamide were combined. There was significantly less MGC formation in the presence of niclosamide, which was maximal at 0.15 μmol/ml. Median aggregate volume PR3 vs PR3 and niclosamide 0.3, 0.15. 0.05. 0.01 μmol/ml respectively 106969 (41031-465184), 42730 (25638-324957), 44501 (26861-90354), 63043 (27395-205060), 117473 (35128-245781) (p<0.05 PR3 vs 0.3 and 0.05, p<0.001 PR3 vs 0.15 μmol/l; one way ANOVA).

The data presented herein clearly demonstrates that inhibitors of the IL-6/STAT3 pathway and IL-6 inhibitors, such as the small molecule niclosamide and the antibody against the human interleukin-6 (IL-6) receptor, Tocilizumab, respectively, act to reduce MGC formation and to prevent granuloma formation. This has been demonstrated in Zebrafish larvae which contain early competent macrophages differentiating in the yolk sac, which have been developed as an established model of granuloma formation, especially in the context of mycobacterial infection. Although they lack T cells early on they can still form macrophage aggregates, typical of granuloma. The novel model recapitulated these macrophage granuloma aggregates after PR3 injection into the yolk sac, with either enzymatically active or inactive PR3. Administration of niclosamide significantly attenuated this macrophage aggregation. Similarly, in cultured monocytes Tocilizumab was demonstrated to reduced granuloma formation.

In Vivo Effect of PR3 on MGC Formation and the Ability of Niclosamide to Reduce MGC Formation In order to understand granuloma formation in vivo, a zebrafish embryo model was developed. Transgenic macrophage reporter zebrafish {Tg (MPEg: cherry)} embryos at 24 hpf, were injected into the yolk sac with human PR3 or Albumin at 1 or 5 μg/ml, either untreated or following heat inactivation. Embryos were imaged at 120 hpf with lightsheet microscopy showing volumes of fused macrophages according to the colour scale (see FIG. 8A-D). Aggregate volumes of macrophages were taken from un-injected fish to establish a normal baseline, which showed a mean 10248 μm³ and SD of 4256 μm³. A significant number of MGCs were observed in injected fish. Low level aggregation in albumin injected animals but the greatest aggregation was following PR3 injection and was observed with equal effect of intact or heat inactivated (HI) PR3 (see FIG. 8E and FIG. 9). In addition, aggregate volume was significantly greater following PR3 injection compared to albumin injection (FIG. 11A) Median (IQR) aggregate volumes were for intact albumin 1 pg/ml 22997 μm³ (20454-32491), HI albumin 1 pg/ml 22005 μm³ (19918-24273), and intact PR3 1 pg/ml 28479 μm³ (22970-48552) and HI PR3 1 pg/ml 31163 (23793-46733) (p<0.05 for intact albumin vs PR3, p<0.001 for HI albumin vs HI PR3, p=ns between albumin and HI albumin or PR3 and HI PR3, one way ANOVA). Higher doses of HI albumin or PR3 did not demonstrate greater MGC formation, but a significant difference was still seen between albumin- and PR3-treated fish. Median (IQR) aggregate volumes were HI albumin 5 pg/ml 22713 μm³ (19918-31486) and HI PR3 5 pg/ml 31571 μm³ (23137-49647) (p<0.001, one way ANOVA). In subsequent experiments, the injection of intact or HI PR3 was repeated in the presence or absence of the IL-6-STAT3 inhibitor niclosamide, added to the fish water at different concentrations (0.01-0.3 μmol/l) (see FIG. 8F-G). Since no difference between PR3 and HI PR3 was noted with respect MGC formation, all the fish injected with PR3 alone and those injected with differing doses of niclosamide were combined. There was significantly less MGC formation in the presence of Niclosamide, which was maximal at 0.15 μmol/ml. Median aggregate volume PR3 vs PR3 and niclosamide 0.3, 0.15. 0.05. 0.01 μmol/ml respectively 106969 (41031-465184), 42730 (25638-324957), 44501 (26861-90354), 63043 (27395-205060), 117473 (35128-245781) (p<0.05 PR3 vs 0.3 and 0.05, p<0.001 PR3 vs 0.15 μmol/l; one way ANOVA).

### Example 3—Reducing MGC Formation Using Stattic

An in vitro model of granulomatous disease was created using THP1 monocytes. The THP1 monocytes were matured to macrophages by the addition of PMA for 24 hours and then stimulated with PR3 at 10 mcg/ml for 72 hours in the presence or absence of Stattic, a STAT3 inhibitor. MGC formation was assessed as before by calculating the fusion index.

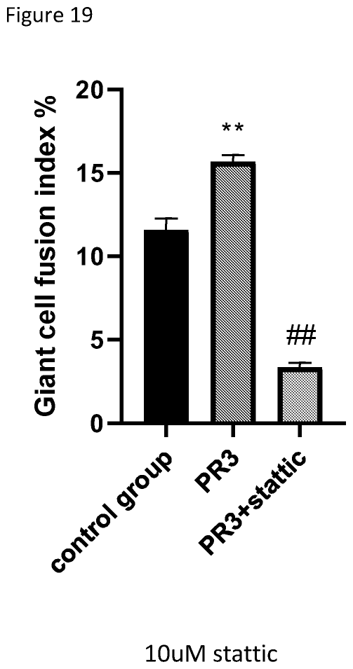

The results are presented in FIG. 19 and demonstrate that there was a significant decrease in giant cell formation index in the cells treated with Stattic relative to the untreated and control cells. This shows that inhibiting STAT3 may be used to reduce giant cell formation and by extension treat a granulomatous disease.

### Methods and Materials

### Methods:

PBMCs were isolated from healthy controls (n=10), GPA (n=10) and microscopic polyangiitis (MPA) (n=10) patients. Monocytes were magnetically isolated. Cells were stimulated with either enzymatically active or inactive PR3 or control auto-antigen myeloperoxidase (MPO) at lug/ml or 10 μg/ml and incubated for 72 hours. Light, epifluorescence and confocal microscopy was used to confirm cell fusion at different time points. Tg (mpx: GFP) zebrafish were anaesthetised at 24 hours post-fertilisation and injected with enzymatically active or inactive PR3 or albumin at 1 pg/ml into the yolk. Fish were imaged by lightsheet microscopy at 7 days post-fertilisation.

### Study Participants

Patients with GPA and MPA were identified from a clinical vasculitis database, with disease classified according to the Chapel Hill Consensus Conference (CHCC) diagnostic criteria. Patient demographics, clinical characteristics and investigations including ANCA reactivity were documented from electronic records. Disease activity, scored by Birmingham Vasculitis Activity Score (BVAS) and Vasculitis Damage Index (VDI) was calculated.

### PBMC and Monocyte Isolation

Blood was collected in EDTA tubes and diluted at a ratio of 1:1 in Dulbecco's phosphate buffered saline (DPBS) without calcium or magnesium (Sigma, UK) and layered over density gradient media Lymphoprep 1.077 g/ml (Alere, UK) at a ratio of 2:1 of diluted blood to density gradient media. Blood was centrifuged at 800×g and peripheral blood mononuclear cell (PBMC) fraction harvested and washed twice in DPBS. Monocytes were subsequently isolated by CD14 positive selection using magnetic bead isolation (Miltenyi Biotec, UK). Isolated CD14 positive cells were re-suspended in complete culture medium and 10% heat inactivated human AB serum (Sigma, UK).

### Monocyte Phenotype Analysis

Monocytes from a subset of PR3-ANCA GPA patients were phenotyped according to CD14 and CD16 expression and percentage frequencies of classical, intermediate, and non-classical subsets calculated by FACS analysis. Percentage CD14 cells expressing PR3, MAC-1 and PAR-2 were then measured. Fluorescence minus one controls were used for gating strategies. Cells were stained with CD14 (BV711 (63D3) Biolegend, UK), CD16 (PerCP/Cy5.5 (3G8) Abcam, UK), PR3 (FITC (WGM2) Abcam, UK), MAC-1 (BV421 (M1/70) Biolegend, UK) and PAR-2 (PE (344222) R&D Biotechne, UK) antibodies and FACS performed on a Fortessa analyser (BD, UK). FlowJo version 7.6.3 (TreeStar, Ashland, OR, USA) was used for data analysis.

### PR3 Heat Inactivation

Chromogen-based enzymatic hydrolysis of N-methoxysuccinyl-Ala-Ala-Pro-Val p-nitroanilide (Sigma, UK) was used to test PR3 enzymatic activity as previously described (see Wiesner O, Litwiller R D, Hummel A M, Viss M A, McDonald C J, Jenne D E, et al. Differences between human proteinase 3 and neutrophil elastase and their murine homologues are relevant for murine model experiments. FEBS Lett. 2005; 579 (24): 5305-12). PR3 1 mg/ml was incubated for 15 minutes at 100° C. to heat inactivate and enzymatic activity tested against enzymatically active PR3. Briefly, 10 μl of PR3 or heat inactivated PR3 at varying concentrations was added to 180 μl 1 mmol N-methoxysuccinyl-Ala-Ala-Pro-Val p-nitroanilide (dissolved in 0.1 mol/L Tris-HCl buffer and 0.5 mol/L NaCl, pH 7.2) and incubated for 24 hours at 37° C. Spectrophotometry was performed at 405 nm and difference in OD values used to test heat inactivation.

### Purification of Human Immunoglobulin

Human anti-PR3 IgG and anti-MPO IgG were isolated from a separate group of PR3-ANCA or MPO-ANCA positive patients, diagnosed with GPA (n=3) or MPA (n=3) respectively or from healthy controls (n=3). Patients were selected for antibody purification based on anti-PR3 or anti-MPO antibody titre, tested in the Royal Free Hospital Clinical Immunology Department as part of routine clinical care. HiTrap Protein G columns (1 ml) (GE Healthcare, UK) chromatography was used to purify antibodies from plasma sample. IgG fractions were buffer exchanged with PBS using commercially available columns (Sartorium Stedin Biotechnology, France) prior to use and concentration measured by spectrophotometry using NanoDrop 8000 (Thermo Scientific, UK). Pre-packed 1 ml Detoxi-Gel™ AffinityPak™ endotoxin removing gel columns (Thermo Scientific Pierce, UK) were used to deplete endotoxin from each isolated IgG sample. To test ANCA reactivity after isolation, indirect immunofluorescence was performed. Samples were incubated with ethanol-fixed neutrophils on glass chamber slides for 20 minutes. Secondary FITC-conjugated mouse anti-human IgG antibody (Dako, UK) was used. Antibody concentration was measured in the Clinical Immunology Laboratory at the Royal Free Hospital using ImmunoCAP 250 (ThermoFisher, UK).

### Monocyte Culture

Experiments followed previously reported methods (see Most J, Spotl L, Mayr G, Gasser A, Sarti A, Dierich MP. Formation of multinucleated giant cells in vitro is dependent on the stage of monocyte to macrophage maturation. Blood. 1997; 89 (2): 662-71 and Gasser A, Most J. Generation of multinucleated giant cells in vitro by culture of human monocytes with Mycobacterium bovis BCG in combination with cytokine-containing supernatants. Infect Immun. 1999; 67 (1): 395-402). Monocytes were cultured in complete medium with or without PR3 (Sigma, UK) in a humidified incubator at 37° C., 5% CO₂ at a density of 1×10⁶ cells/mL in glass chamber slides, each condition was tested in duplicate. Complete medium was a negative control. Culture medium supernatant was removed after incubation, centrifuged and stored at −80° C. for cytokine analysis. Stimuli used throughout MGC formation experiments are outlined and were used in isolation or in combination with PR3.

Concanavalin A (ConA) was used to establish and confirm MGC formation. PBMCs from healthy controls were isolated and stimulated with ConA 16 μg/ml for 72 hours. Cytokine-rich supernatants were then collected and used at 50% concentration in complete medium for initial monocyte culture experiments. Human PR3 (Sigma, UK), MPO and elastase (Calbiochem, Merck, UK) were extracted from human neutrophil polymorphonuclear cells. Heat inactivation resulted in loss of PR3 enzymatic activity (HiPR3).

Inhibition of MGC formation was tested by culturing monocytes in the presence of anti-MAC-1 antibody (10 μg/mL) or control immunoglobulin (10 μg/ml) (Abcam, UK). The effect of humanised IgG1 monoclonal antibody against the human interleukin-6 (IL-6) receptor, Tocilizumab (RoActerma, Roche, UK) was tested a concentration of 10 μg/mL.

### Calculation of Monocyte Fusion Index

Samples were stained with a modified Giemsa stain (0.4% w/v, in a buffered methanol solution, pH 6.9) (Sigma, UK). Light microscopy was performed using a Nikon Eclipse Ci camera and Nikon DS-L3 software (Amstelveen, Netherlands). Giemsa stained images were analysed using ImageJ software (Fiji). For every sample, each well was imaged at ×100 magnification, converted to 8-bit type and switched to grayscale. Colour threshold was automatically applied and not altered by the operator to minimise bias. Images were reset in black and white and binary ‘watershed’ function used to recognise separation in pixel values between areas of aggregation allowing accurate quantification of percentage fused cells per surface area. Representative MGC size was determined by scanning electron microscopy (SEM) and average (n=3) value used as size threshold (greater than 37 μm²).

### Scanning Electron Microscopy

Scanning electron microscopy was used to assess the ultrastructure and surface topography of fused cells. Monocytes stimulated with PR3 were used to evaluate MGC formation. Cells were cultured on 13 mm glass coverslips, and fixed with glutaraldehyde. Images were acquired using a Carl Zeiss EVO HD LS15 with SmartSEM software version 5.07.

### Confocal Laser Microscopy

PR3 stimulated cells were cultured on glass chamber slides and stained with wheat germ agglutin (WGA) Alexa Fluor 594 (ThermoFisher, UK), FITC-phalloidin (SantaCruz, USA) and 4′,6-diamidino-2-phenylindole (DAPI) (Abcam, UK). Cells were permeabilised with 0.1% Triton-X100 (Sigma, UK) then stained with WGA, phalloidin and DAPI according to the manufacturer's recommendations. Slides were mounted with Vectashield fluorescent mounting medium (Vector). Confocal laser scanning microscopy was performed on Leica SP5 and SP2 microscopes. Images were processed and analysed using Infinity Capture and Analyze V6.2.0, ImageJ 1.50h75 and the Leica Application Suite, Advanced Fluorescence 3.1.0 build 8587 Software.

### Cytokine Analysis

Cytokine cytometric bead assays (LegendPLEX, Biolegend, UK) were performed to measure levels of individual cytokines in culture supernatants, according to the manufacturer's instructions. Bead populations were gated, identified on FSC and SSC. APC was used as the classification channel and PE and FITC as the reporter channels. Median fluorescence intensity and cytokine/chemokine concentrations were calculated. Culture supernatant IL-6 concentration was further tested by a commercially available ELISA (R&D, UK).

In Vivo Danio rerio Model All animal experimental protocols were approved by University College London's Animal Welfare and Ethical Review Body (AWERB) and licensed by the UK Home Office. All experiments were performed in accordance with relevant guidelines and regulations as described in UK Home Office Project License (PPL 70/8365) awarded to Dr Paul Frankel. The Transgeneic Tg (mpeg: GFP) line was kindly provided by Professor Maggie Dallman (Imperial College London, UK). Zebrafish were maintained in mixed-sex populations at 28° C. with a 14 h: 10 h light: dark cycle from 9 am-11 μm and within internationally agreed environmental parameters regarding water quality and composition. Following dechorionation and anaesthesia with tricaine (0.2 mg/ml), embryos were microinjected into the yolk sac with 5 pg/mL of PR3, heat inactivated PR3, or albumin and incubated in 10 cm Petri dishes at 28.5° C. After 5 days, living embryos were anaesthetised with tricaine (0.2 mg/ml) and embedded in 2% agarose for 3-dimensional confocal microscopy using a Ziess camera at ×200 magnification to determine macrophage fusion. Z-stacks were analysed using Imaris software version 9.2. Images were 3D rendered and aggregate volumes measured by automated thresholds.

### Statistics

Statistics were performed using GraphPad Prism software version 8. Categorical variables were tested using Chi-squared tests. Median values with 95% confidence intervals were reported. Values calculated included mean, standard error of the mean (SEM), standard deviation (SD), median, interquartile range (IQR) and percentage. Rates of monocyte fusion were tested by Mann-Whitney U tests for two variables or by one or two way ANOVA for three or more. Significance was defined by p value <0.05.

### Study Approval

Research described was in accordance with the Declaration of Helsinki and approved by the NHS Research Ethics Committee (05/Q0508/6). In addition, patients were recruited to the ‘UK vasculitis susceptibility and patient outcomes study’ (10/H1102/77 NRES London). University College London ethics committee approved animal experiments.

Methods—PBMCs were isolated from healthy controls (HC) (n=16), PR3-ANCA GPA (n=16), MPO-ANCA GPA (n=3) and MPO-ANCA MPA (n=16) remission patients and stimulated for 72 hours with or without PR3 (10 μg/ml), MPO (10 μg/ml) or control conditioned medium. Cells were stained and analysed by Bright field, confocal immunofluorescence and scanning electron microscopy (SEM) to demonstrate aggregation and fusion. Cytokine production was quantified by CBA and ELISA.

### Subjects

A total of 34 GPA patients (30 with PR3-ANCA and 4 with MPO-ANCA), 10 MPA patients (all with MPO-ANCA) and 10 healthy controls were recruited and venesected. Demographics were recorded for all patients. Birmingham Vasculitis Activity Score (BVAS) and Vasculitis damage index (VDI) was recorded for each patient and specifically showed more ocular, ENT and pulmonary manifestations in GPA patients compared to renal involvement in MPA patients. Immunosuppression using prednisolone and azathioprine were used most frequently in GPA patients.

1. An interleukin-6 (IL-6)/signal transducer and activator of transcription 3 (STAT3) pathway inhibitor for use in treating a granulomatous disease in a subject.

2. The IL-6/STAT3 pathway inhibitor for use according to claim 1, wherein the inhibitor is an IL-6 inhibitor.

3. The IL-6/STAT3 pathway inhibitor for use according to claim 1, wherein the inhibitor is a STAT3 inhibitor.

4. The IL-6/STAT3 pathway inhibitor for use according to claim 1, wherein the inhibitor is niclosamide or a salt thereof.

5. The IL-6/STAT3 pathway inhibitor for use according to claim 1, wherein the inhibitor is an anti-IL-6 antibody or fragment thereof.

6. The IL-6/STAT3 pathway inhibitor for use according to any one of claims 1-5, wherein the granulomatous disease is Antineutrophil Cytoplasmic Antibodies (ANCA) associated vasculitis, Crohn's disease, or sarcoidosis.

7. The IL-6/STAT3 pathway inhibitor for use according to claim 6, wherein the ANCA associated vasculitis is granulomatosis with polyangiitis or eosinophilic granulomatosis with polyangiitis.

8. A method of treating a granulomatous disease in subject comprising administering an IL-6/STAT3 pathway inhibitor to the subject.

9. The method according to claim 8, wherein the IL-6/STAT3 pathway inhibitor is an IL-6 inhibitor.

10. The method according to claim 8, wherein the IL-6/STAT3 pathway inhibitor is a STAT3 inhibitor.

11. The method according to claim 8, wherein the IL-6/STAT3 pathway inhibitor is niclosamide or a salt thereof.

12. The method according to claim 8, wherein the IL-6/STAT3 pathway inhibitor is an anti-IL-6 antibody or fragment thereof.

13. The method according to any one of claims 8-12, wherein the granulomatous disease is Antineutrophil Cytoplasmic Antibodies (ANCA) associated vasculitis, Crohn's disease, or sarcoidosis.

14. The method according to claim 13, wherein the ANCA associated vasculitis is granulomatosis with polyangiitis or eosinophilic granulomatosis with polyangiitis.

15. Niclosamide or a salt thereof for use in treating a granulomatous disease in a subject.

16. A method of treating a granulomatous disease in subject comprising administering niclosamide or a salt thereof to the subject.

17. Niclosamide or a salt thereof for use according to claim 15, or the method of claim 16, wherein the granulomatous disease is Antineutrophil Cytoplasmic Antibodies (ANCA) associated vasculitis, Crohn's disease, or sarcoidosis.

18. Niclosamide or a salt thereof for use according to claim 17, or the method of claim 17, wherein the ANCA associated vasculitis is granulomatosis with polyangiitis or eosinophilic granulomatosis with polyangiitis,

19. An anti-IL-6 antibody or fragment thereof for use in treating a granulomatous disease in a subject.

20. A method of treating a granulomatous disease in subject comprising administering an anti-IL-6 antibody or fragment thereof to the subject.

21. The anti-IL-6 antibody or fragment thereof for use according to claim 19, or the method of claim 20, wherein the granulomatous disease is Antineutrophil Cytoplasmic Antibodies (ANCA) associated vasculitis, Crohn's disease, or sarcoidosis.

22. The anti-IL-6 antibody or fragment thereof for use according to claim 21, or the method of claim 21, wherein the ANCA associated vasculitis is granulomatosis with polyangiitis or eosinophilic granulomatosis with polyangiitis.
